(19) United States
(12) Patent Application Publication
(10) Pub. No.: US 2026/0283938 A1
(43) Pub. Date: Sep. 24, 2026

(54) Cosmetic And Method of Preparing Cosmetic Compositions with 100 Times Washed 100 A2 Ghee

(71) Applicant: PSR Global Consultancy Inc., Fort Lee, NJ (US)

(72) Inventor: Pushker Sona, Fort Lee, NJ (US)

(73) Assignee: PSR Global Consultancy Inc., Fort Lee, NJ (US)

(21) Appl. No.: 19/573,393

(22) Filed: Mar. 20, 2026

Related U.S. Application Data
(60) Provisional application No. 63/775,373, filed on Mar. 21, 2025.

(51) Int. Cl. A61K 8/92; A61Q 19/08

(52) U.S. Cl. CPC A61K 8/925; A61Q 19/08; A61K 2800/21; A61K 2800/805

(57) ABSTRACT

The use of ghee-based lipids in cosmetic compositions presents technical challenges including poor compatibility with aqueous systems, phase instability, oxidation, and undesirable odor, which limit formulation options and commercial applicability. Disclosed herein are processes and cosmetic compositions that address these challenges by converting A2 ghee into a form suitable for incorporation into aqueous cosmetic systems. In example processes, A2 ghee is liquefied, repeatedly washed with water in a copper vessel through multiple cycles, including up to one hundred repetitions, and separated to remove impurities, thereby producing washed A2 ghee having improved physical characteristics. The washed A2 ghee is subsequently combined with cosmetic additives to form stable cosmetic compositions, including oil-in-water emulsions, surfactant-based compositions, and suspensions. The disclosed enable stable dispersion of washed A2 ghee in cosmetic formulations with improved physical stability and sensory acceptability, providing a technically robust solution for incorporating ghee-derived lipids into topical cosmetic products.

### TECHNICAL FIELD

Disclosed is a composition and method for preparing a range of health care and skin care compositions made with 100 times washed A2 Ghee also known as South Asian clarified butter. The compositions are useful, among other things, for moisturizing, hydration, nourishing, and skin protectant benefits. The composition may also assist in delayed aging, scar prevention, and skin radiance. More particularly, disclosed is a cosmetic composition and preparation method of various formulations utilizing clarified butter fat.

### BACKGROUND

It is well known that Ghee is a very pure type of fat made from simmering butter until the fat, also known as clarified butter, separates from the milk solids. It has also been called by some as the most refined end product of milk. Ghee originated in India around 3,000 years ago and is now frequently used in South Asian and Middle Eastern cuisines for its high “smoke point” and because of its stability unrefrigerated. Ghee contains various nutrients including Vitamins A, D, K, and E among other things.

Also Ghee is known to deeply moisturize and nourish the skin, leaving it soft, supple, and glowing. The rich vitamins and antioxidants present in ghee help in repairing damaged skin cells and reducing signs of aging. Regular application of ghee on the face can improve skin elasticity and promote a youthful appearance. Again ghee is a type of clarified butter. The whole process remains the same while making cow ghee but instead of removing the butter from the heat once the milk solids separate, it is left to cook a little while longer. When the proteins turn golden brown and start giving off a nutty aroma, and is strained and stored.

However, removing butters and clarified butters from foods such as milk can be expensive and may reduce various characteristics from the ghee. Therefore, it would be desirable to provide a fat based concentrate with improved characteristics that can be used in smaller quantities than traditional butters and clarified butters to improve the nutritional properties without a loss of vital properties. Furthermore, good quality ghee is expensive compared to presently available imitations. It would be desirable to provide cost-effective alternatives to high quality ghee, preferably without sacrificing desired characteristics.

Therefore, there still exists a critical need for a novel 100× ghee prepared from A2 variety of Ghee and cosmetic composition that allows for effective treatment without loss of vital properties and without adverse effects. In addition, there is a need for a methodology to cost-effectively provide high quality 100× washed ghee prepared from A2 variety of Ghee in various formulations.

### SUMMARY OF THE INVENTION

Compared to the above prior attempts, the presently disclosed composition and method solves the problems of current state of the art, meets the above requirements, and provides many more benefits.

The present invention relates to personal care compositions made with 100 times washed A 2 Ghee in an oil in water (O/W) emulsion, suspension, ointment, or Jelly or Liquid soap/surfactant and an oil. The O/W emulsion is composed of an oil phase dispersed in an aqueous phase also known as a direct emulsion. Stabilization of O/W the emulsion is performed with hydrophilic-hydrophobic particles.

In an embodiment, the invention is a composition of skin care formulations comprising (a) 100 times washed A2 Ghee (b) water and (c)] other cosmetic ingredients in a range of formats such as but not limited to creams, lotions, serums, lip balm and masks.

A2 ghee means a particular variety of clarified butter derived from cows whose milk naturally contains the A2 beta-casein protein. This protein is found in the milk of cows that are descended from the ancient Indian cow breeds such as Gir, Sahiwal, and Red Sindhi. A2 ghee benefits are countless and hence is considered a superfood by many. It is rich in vitamins, minerals, and antioxidants and is also known to have anti-inflammatory properties. A2 ghee is widely used in Ayurvedic medicine and is believed to have a positive impact on digestion, immunity, and overall well-being. A2 ghee is also considered an excellent source of healthy fats and is known to aid in weight loss, improve brain function, and boost energy levels. As macrobiotic cow ghee is packed with fat-soluble vitamins that not only aid weight loss but also play a key role in balancing hormones and maintaining healthy cholesterol levels. A2 cow ghee also has a high heat point preventing the production of free radicals that can damage cell function.

The present invention also relates to combining functional actives in a golden ratio resulting in improved compositions.

The above objects and advantages are met by the present invention. In addition, the above and yet other objects and advantages of the present invention will become apparent from the hereinafter set forth. Brief Description of the Drawings, Detailed Description of the Invention and claims appended herewith. These features and other features are described and shown in the following drawings and detailed description.

### BRIEF DESCRIPTION OF FIGURES

So that those having ordinary skill in the art will have a better understanding of how to make and use the disclosed composition and methods, reference is made to the accompanying figures wherein:

FIG. 1. is a schematics of one embodiment of the proposed invention.

FIG. 2 is a process flow diagram illustrating example methods for forming cosmetic compositions using 100 times washed A2 ghee produced according to one embodiment of the process utilizing one aspect of the disclosure of the invention shown in FIG. 1.

### DETAILED DESCRIPTION

The present disclosure is directed to a new composition, a process, and novel strategy for making personal care compositions made with 100 times washed A 2 Ghee in an oil in water (O/W) emulsion, suspension, ointment, or Jelly or Liquid soap/surfactant and an oil.

The compositions and processes described herein are not intended to diagnose, treat, cure, or prevent any disease, and any references to traditional or historical uses are provided solely for background context and not as therapeutic claims. The compositions disclosed herein are intended for cosmetic and personal-care use, including improving skin feel, hydration, softness, and appearance, supporting the skin barrier, and providing conditioning and moisturizing effects. The disclosed formulations are designed to exhibit physical stability, reduced odor, oxidation resistance, and favorable sensory properties, making them suitable for use in creams, lotions, emulsions, cleansers, and suspensions applied to healthy skin.

Regular ghee is produced by mixing the milk of buffalo and cows. These animals are mostly from the European region and are mostly crossbred. The source of the milk, the breed of the cow and the overall traceability of the product are never known to customers. Many questions are never answered to consumers such as, what do the cows consume, whether any hormone injections given to them, how is their lifestyle, are they sent to the slaughterhouse once they stop giving milk, and other such questions. All such information is never provided to the customer. This ghee is made on an industrial level in huge quantities. Many times, additives like color.

Again, A2 ghee is produced from the milk of an indigenous breed cows [For this invention a particular breed of cattle known as the Gir cow was used as a source of A2_. These cows are native to the Gir forests in Gujarat with full record of traceability. Genuine A2 Ghee is made in gaushalas/farms in smaller batches. No additives are present in this ghee which results in the product being different in each batch. The cows are not injected with hormones and are provided with organic fodder only. The sellers specify all this information if they are selling genuine A2 Ghee. The A2 Ghee is ideally made using the Bilona process of making ghee which involves making Makhan (butter) from curds and not from malai (cream).

The milk from these cows naturally produces the A2 beta-casein protein. Cow milk contains both A1 beta-casein protein and A2 beta-casein protein. But some modern researchers have underlined that A2 protein is healthier than A1 protein. It is believed that the milk of Desi cow has more A2 protein. Hence, they are present in the ghee obtained by A2 cow milk if the cows are grass-fed along with other nutrition-rich foods.

### Purity

Regular ghee is produced on a mass-scale at industrial levels. At times, the cows are injected with hormones to produce more milk. A2 Gir cow ghee is produced in small batches on cow farms. Here, the cows are not given any hormones to increase their milk production. Instead, they are fed with organic fodder to keep them healthy. Since A2 Gir cow ghee is produced through a traditional process without the use of machinery and excessive heat, it retains most of its nutrition.

Ayurveda has professed that the ghee prepared from A2 cow milk using the traditional bilona or valona process contains Omega-3 and -9. It is a rich source of unadulterated anti-oxidants, vitamin A, a superior quality protein and a variety of micronutrients that help build immunity. Pure cow ghee is a part of several Ayurvedic medicines which explains its significant role in maintaining health. A2 ghee emerges as a treasure trove of fat-soluble vitamins-A, D, E, and K. This rich vitamin cache fortifies immune function, bolsters bone health, safeguards vision, nurtures skin, and aids in blood clotting. Its fat-soluble nature ensures optimal absorption, harnessing these vital nutrients for the body's benefit.

### What are the Health Benefits of A2 Cow Ghee?

It helps heal wounds faster. For thousands of years in Ayurveda, ghee has been used to treat and heal cut and burn wounds. It has anti-inflammatory properties, which soothe the skin and prevent infections while helping the wounds heal quicker. It keeps your skin beautiful. One of the most important benefits of A2 Gir cow ghee is that it protects your skin from the damage caused by free radicals. These free radicals are responsible for making your skin look aged by causing pigmentation, wrinkles, and age spots.

Applying ghee to the skin and consuming it regularly prevents this and keeps your skin looking youthful for longer.

### 100 Times Washed Ghee-how is the Ghee Washed 100 Times?

It is done by placing ghee into a copper vessel and mixing it with purified water literally one hundred times while chanting sacred mantras. The water is added to ghee, everything mixed for 3-5 minutes, then water is discarded, washing the ‘impurities’ away from the butter. The result is an odorless cream of whipped butter consistency According to instructions found in the 400-200 BC Charaka Samhita, one of the greatest texts on Ayurveda, this cream is to be made on the night of the full moon in a pure copper vessel by washing pure A2 cow ghee 100 times while reciting sacred mantras. By using this technique, the substance becomes lighter, more voluminous, and has smaller particles, making it easier for the skin to absorb. The cleaning of ghee incorporates the benefits of copper metal, copper utensils were employed. This minimal amount of copper may aid in the synthesis of elastin and collagen, which can lessen wrinkles and fine lines around the skin. Copper has strong antioxidant and antibacterial properties.

### The Chemistry of 100-Times Washed Ghee

It starts as a lipid (fatty) emulsion and with each introduction of rinsing water, as the pressure from mixing gets applied to fat particles, it splits them and makes the emulsion more aqueous (water infused). This ‘fat splitting is the process in which, fat is hydrolyzed in the presence of water to yield free fatty acids and glycerol's (source). Copper acts like a catalyst to promote fat splitting, and also increases the copper content in the emulsion. Plus copper has anti-inflammatory effect on skin (source).

Benefits of 100 times washed A2 Ghee

Ghee that has been washed 100 times aids in skin whitening, hydrating, delaying wrinkles and age spots, and many other benefits. Omega 3, 9 fatty acids, and vitamins A, D, E, and K are all found in Shata Dhauta Ghrita and are all said to be excellent for skin health. Dec. 8, 2023 Ayurveda refers to 100 times-washed ghee as Shata Dhauta Ghrita, where “Shata” means “hundred,” “Dhauta” means “washed,” and “Ghrita” means “Ghee.” that the Ayurvedas believe Due to its small particle size, it may be able to deliver pharmaceutical ingredients deep into skin tissue reach through all seven layers of the skin without blocking pores.

It is believed to be an excellent anti-aging treatment that smooths wrinkles, fades sunspots, heals burns, soothes inflammation of rosacea, eczema, acne, and the like.

The below discussion and definitions are intended to guide understanding but are not intended to be limiting with regard to other disclosures in this application. Throughout this application, references to percentage (%) of compositions of the present invention refers to the % by weight of a given substance to the total weight of the composition being discussed, also signified by “w/w” or “wt/wt”, unless stated otherwise.

For purposes of this disclosure, the following terms shall have the meanings set forth below unless the context clearly indicates otherwise. “About” refers to a variation of ±10% of a stated numerical value, and in certain embodiments ±5% or ±2%, consistent with normal manufacturing and measurement tolerances. “A2 ghee” refers to clarified butter derived from milk of cows whose milk naturally contains the A2 beta-casein protein, including milk from breeds such as Gir, Sahiwal, and Red Sindhi, and equivalents thereof.

The term “100 times washed A2 ghee” or “washed A2 ghee” refers to A2 ghee that has been repeatedly combined with water and mixed, followed by removal of the water, for a plurality of cycles up to one hundred cycles, optionally performed in a copper vessel, resulting in a cream-like lipid material suitable for cosmetic formulation.

“Cosmetic composition” refers to a composition intended for topical application to skin for cosmetic or personal-care purposes, including improving skin feel, hydration, appearance, conditioning, or barrier support, and not for diagnosing, treating, curing, or preventing disease. “Effective amount” refers to an amount of a component sufficient to provide a desired cosmetic effect, such as moisturizing, hydration, nourishment, barrier support, or conditioning, without causing unacceptable sensory characteristics or instability. “Excipient” refers to a cosmetically acceptable ingredient other than the primary active lipid (e.g., washed A2 ghee) that facilitates formulation, stability, performance, or aesthetics of the cosmetic composition, including but not limited to water, oils, surfactants, emulsifiers, thickeners, humectants, preservatives, fragrances, and combinations thereof.

“Humectant” refers to a cosmetically acceptable ingredient that attracts and retains water in the outer layers of the skin, thereby providing hydration and moisturizing effects. “Emollient” refers to a cosmetically acceptable lipid or oil-based ingredient that softens, smooths, or conditions the skin surface by improving skin feel and flexibility. “Occlusive” refers to a cosmetically acceptable ingredient that forms a film or barrier on the skin surface to reduce transepidermal water loss and support cosmetic barrier function. “Conditioning agent” refers to a cosmetically acceptable ingredient that enhances tactile properties, spreadability, slip, smoothness, or overall sensory performance of a cosmetic composition. “Barrier support” or “barrier protection” refers to cosmetic effects associated with reducing moisture loss and supporting the integrity of the skin's outer layer, without implying medical repair or treatment.

The term “Oil-in-water (O/W) emulsion” refers to a cosmetic composition in which an oil phase is dispersed within a continuous aqueous phase. “Hydrophilic-hydrophobic particles” refers to solid particles having both hydrophilic and hydrophobic surface characteristics capable of stabilizing an oil-in-water emulsion by adsorption at the oil-water interface.

The terms “Particle-stabilized emulsion” refers to an emulsion in which dispersed phase stability is provided at least in part by hydrophilic-hydrophobic particles rather than, or in combination with, conventional surfactants or emulsifiers.

“Surfactant system” refers to one or more surfactants selected from anionic, non-ionic, amphoteric, or combinations thereof, formulated to provide cleansing, dispersion, or conditioning properties suitable for cosmetic use. “Suspension” refers to a cosmetic composition in which solid or particulate materials are dispersed within a liquid phase and maintained in a substantially uniform distribution through the use of thickening or suspending agents. “Cosmetically acceptable” refers to ingredients and compositions that are suitable for topical application to skin, compatible with other formulation components, and acceptable from a sensory and safety standpoint for cosmetic use.

As used herein, the term “submicron” range refers to particles or droplets having an average size of less than about 1 micrometer (μm), including sizes from about 0.1 μm to less than 1.0 μm. The term “low-micron” range refers to particles or droplets having an average size in the range of about 1 μm to about 5 μm. The term “micron” refers to particles or droplets having an average size in the range of about 1 μm to about 10 μm.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Again the use of the term “about” is intended to describe values either above or below the stated value in a range of approximately +10%; in other embodiments, the values may range in value above or below the stated value in a range of approximately +5%; in other embodiments, the values may range in value above or below the stated value in a range of approximately +2%; in other embodiments, the values may range in value above or below the stated value in a range of approximately +1%. The preceding ranges are intended to be made clear by context, and no further limitation is implied.

The use of any and all examples, or exemplary language (e.g., “such as”) provided herein, is intended merely to better illuminate the invention, and does not pose a limitation on the scope of the invention unless otherwise stated. No language in the specification should be construed as indicating any non-claimed element as essential to the practice of the invention.

In various embodiments, the present disclosure relates to cosmetic and personal-care compositions comprising 100 times washed A2 ghee, optionally combined with water and one or more cosmetically acceptable ingredients, to provide cosmetic benefits such as moisturization, hydration, nourishment, barrier support, and conditioning of skin. The compositions may be formulated in multiple product formats including, without limitation, oil-in-water (O/W) emulsions, suspensions, ointments, jellies, and liquid soap or surfactant-based compositions.

Again as used herein, “A2 ghee” refers to clarified butter derived from cows whose milk naturally contains the A2 beta-casein protein, including milk from breeds such as Gir, Sahiwal, and Red Sindhi. In certain embodiments, the A2 ghee used herein is sourced from Gir cows with traceability and prepared in smaller batches, optionally using a traditional bilona process. In certain embodiments, “100 times washed A2 ghee” (also referred to as Shata Dhauta Ghrita) is prepared by repeatedly combining ghee with water and mixing, followed by removal of the water, for a plurality of cycles up to one hundred cycles. In an example preparation method, ghee is heated until it becomes a liquid, the liquid ghee is placed in a copper vessel, cool water is added and mixed for about 3-5 minutes, the water is poured off and/or strained, and the steps are repeated 100 times, optionally followed by addition of one or more cosmetic actives.

In certain embodiments, the repeated washing process alters physical characteristics of the ghee, for example forming a cream-like consistency and reducing odor, and may incorporate effects associated with contact with a copper vessel. The resulting washed ghee can then be incorporated into the cosmetic compositions described herein in amounts effective for cosmetic performance.

In certain embodiments, the compositions further comprise one or more cosmetically acceptable excipients or auxiliaries selected from water, oils, emulsifiers, surfactants, thickeners, suspending agents, chelating agents, preservatives, fragrances, essential oils, extracts, vitamins, and combinations thereof. For example, in certain embodiments a liquid phase may include one or more of silicone(s), humectant(s), emollient(s), water, vegetable oil(s), synthetic oil(s), fragrance, extract(s), sorbitan oleate, and/or isopropyl myristate. In certain embodiments, the oil phase may include fatty acids, esters, thickening agents such as fatty alcohols, vegetable butters, alcohol benzoates, and antioxidants, and the composition may include vitamins such as vitamin A, vitamin C, vitamin B3, and vitamin E.

In certain embodiments, the cosmetic composition comprises one or more ingredients selected to provide one or more cosmetic effects such as moisturizing, nourishing, hydration, barrier support, and conditioning benefits. Such ingredients may be included in amounts suitable for the intended product format and may be combined with the 100 times washed A2 ghee in an effective amount.

In certain embodiments, the cosmetic composition is an oil-in-water (O/W) emulsion comprising an aqueous phase and an oil phase dispersed therein, where the oil phase includes an effective amount of 100 times washed A2 ghee. In such embodiments, stabilization of the O/W emulsion is performed using hydrophilic-hydrophobic particles, thereby forming a particle-stabilized emulsion suitable for topical cosmetic application.

In certain embodiments, the O/W emulsion format may be provided as a cream, lotion, serum, or related cosmetic product. The composition may include, in addition to the washed A2 ghee and water, one or more of emollients, anionic emulsifiers, non-ionic emulsifiers, humectants, actives, chelating agents, preservatives, and essential oils, and may be formulated so that the dispersed phase remains stable during normal storage and use conditions.

In certain embodiments, an O/W emulsion composition comprises (by weight, based on total composition weight): emollients in an amount of about 1-10%, anionic emulsifiers in an amount of about 1-5%, non-ionic emulsifiers in an amount of about 1-5%, humectants in an amount of about 1-10%, actives in an amount of about 0.1-2%, 100 times washed A2 ghee in an amount of about 1-10%, chelating agent in an amount of about 0.05-1%, preservatives and/or essential oils in an amount of about 0.5-2%, and water as much as enough close to 100% (after all other ingredients are accounted for, so the final formula totals 100%).

In certain embodiments, the cosmetic composition comprises a surfactant-based formulation such as a liquid soap or cleanser and includes a surfactant system comprising anionic surfactant(s), non-ionic surfactant(s), and amphoteric surfactant(s) together with an effective amount of 100 times washed A2 ghee. In such embodiments, the surfactant system may be selected to provide cleansing performance while maintaining a cosmetically acceptable feel and conditioning effect attributable, at least in part, to the presence of the washed A2 ghee and optional cosmetic auxiliaries.

In certain embodiments, the surfactant-based composition further includes one or more additional oils, including blends of natural vegetable oils with the washed A2 ghee, and may further include fragrances, extracts, essential oils, silicone(s), and other cosmetically acceptable ingredients for consumer-acceptable sensory characteristics.

In certain embodiments, the surfactant-based composition is formulated to a pH suitable for topical cosmetic use, and the surfactant system is selected to maintain dispersion or distribution of the washed A2 ghee within the product during storage and use.

In certain embodiments, the cosmetic composition is formulated as a suspension comprising water, an effective amount of 100 times washed A2 ghee, and one or more powders and/or particulate components in combination with one or more thickening or suspending agents to maintain a suspended structure. In certain embodiments, the powder component comprises one or more of thickening and suspending agents such as kaolin, xanthan gum, veegum, rice starch, and titanium, and the suspension may be used in cosmetic formats including masks or topical pastes.

In certain embodiments, additional ingredients used in the suspension may include natural actives such as turmeric powder, sandalwood powder, honey, mustard oil, avocado oil, and vitamin B3. In certain embodiments, the suspension further includes one or more nonionic emulsifiers such as polysorbate 20, sorbitan laurate, and PEG-100 stearate to facilitate dispersion of lipids and/or actives within the suspension.

In certain embodiments, the suspension composition comprises (by weight, based on total composition weight): clay in an amount of about 10-40%, thickening agent in an amount of about 1-5%, emulsifiers in an amount of about 1-5%, emollients in an amount of about 5-15%, humectants in an amount of about 5-15%, water as much as is enough close to 100% (after all other ingredients are accounted for, so the final formula totals 100%), and 100 times washed A2 ghee in an amount of about 1-10%.

In certain embodiments, the cosmetic composition is formulated as an ointment or jelly and includes 100 times washed A2 ghee in combination with one or more waxes, oils, and vegetable butters to provide a cosmetically acceptable topical base. Such embodiments may be configured for leave-on application and may further include selected actives and/or vitamins as described herein.

In certain embodiments, the compositions are intended for cosmetic use to improve skin feel, hydration, and appearance and are formulated and described in terms of cosmetic performance (e.g., stability, spreadability, conditioning feel) rather than medical treatment. References herein to effects such as moisturizing, nourishing, hydration, barrier support, and conditioning are intended as cosmetic effects associated with topical application to skin.

In an embodiment, a process of the present invention comprises, consists essentially of, or consists of 100% washed A2 Ghee in a range of personal care composition essentially of, or consists of creams, lotions, suspensions, or ointments.

In an embodiment of this invention, one of the phases may be one or more of a solubilizing agent, suspending agent, and/or carrier. For instance, in an embodiment of this invention, a liquid may comprise silicone(s), humectant(s), emollient(s), water, vegetable Oil(s), synthetic oil(s), and other such substances.

In an embodiment, a liquid used in a Liquid Phase of this invention comprises, consists essentially of, or consists of a mixture of fragrance, extract(s), and silicone(s).

In an embodiment, a liquid used in a Liquid Phase of this invention comprises, consists of, or consists essentially of a mixture of, silicone, sorbitan oleate, isopropylmyristate, fragrance, and essential oil(s). In an embodiment, oil phase may contain, fatty acids, esters, thickening agents such as fatty alcohols, vegetable butters, Alcohol benzoates, antioxidants etc. In an embodiments, composition may contain a range of vitamins such as Vit A, Vit C, Vit B3 and Vit E.

A “powder” e.g. as used in a suspension according to the present invention, in an embodiment, comprises thickening and suspending agents such as Kaolin, Xanthan Gum Veegum and rice starch, and Titanium.

In an embodiment, other ingredients used in the suspension formulation may contain natural actives such as Turmeric powder, Sandalwood powder, Manuka Honey, Mustard oil, Avocado oil and Vitamin B3

In an embodiment of this invention, suspension may contain nonionic emulsifiers such Polysorbate 20, Sorbitan laurate and PEG 100 stearate.

In an embodiment of this invention, ointment may include waxes, oils, and vegetable butters along with 100 times washed A2 Ghee.

The present invention may be further understood in connection with the following Examples and embodiments. The following non-limiting Examples and also embodiments described throughout this application are provided to illustrate the invention.

### Examples

### Example 1: [O/W Emulsion]

| Ingredient | Amount |
|---|---|
| Emollients | 1-10% |
| Anionic emulsifiers | 1-5% |
| Non ionic emulsifiers | 1-5% |
| Humectants | 1-10% |
| Actives | 0.1-2% |
| 100 times Washed A2 Ghee | 1-10% |
| Chelating agent | 0.05-1% |
| Preservatives Essential oils | 0.5-2% |
| DM Water | QS to 100 |

### Example 2 [Suspension]

| Ingredient | Amount |
|---|---|
| Clay | 10-40% |
| Thickening Agent | 1-5% |
| Emulsifiers | 1-5% |
| Emollients | 5-15% |
| Humectants | 5-15% |
| DM Water | QS to 100 |
| 100 times Washed A2 Ghee | 1-10% |

In certain embodiments, the cosmetic composition further comprises at least one ingredient selected from humectants, emollients, occlusives, conditioning agents, or combinations thereof, wherein such ingredient provides one or more effects selected from moisturizing, hydration, nourishing, barrier protection, and skin conditioning benefits.

Suitable humectants may include, by way of example and not limitation, glycerin, sorbitol, sodium PCA, hyaluronic acid, urea, aloe vera, or combinations thereof, which function to attract and retain water within the outer layers of the skin. Suitable emollients may include lipid-based materials such as natural oils, esters, fatty acids, fatty alcohols, silicone oils, vegetable butters, clarified butter derivatives, and combinations thereof, which function to soften, smooth, and condition the skin surface. Suitable occlusives may include waxes, squalane, lanolin, beeswax, silicone-based film formers, lipid-rich fats, or combinations thereof, which function to reduce transepidermal water loss and support the cosmetic barrier function of the skin. Suitable conditioning agents may include fatty esters, silicone derivatives, botanical oils, lipid-based conditioning agents, surfactant-compatible conditioners, or combinations thereof, which enhance spreadability, skin feel, and overall cosmetic elegance of the composition.

In certain embodiments, 100 times washed A2 ghee functions as one or more of an emollient, conditioning agent, and barrier-supporting lipid within the cosmetic composition, optionally in combination with one or more additional humectants, occlusives, or conditioning agents to provide a balanced moisturizing system suitable for topical cosmetic application.

Using one or more of the above ingredients provides the following effects selected from a group consisting of: moisturizing, nourishing, hydration, barrier protection, conditioning benefits, and any combination thereof. The cosmetic composition of this invention may be used as alone or as a regime with other compatible compositions.

Based on the above Examples the following samples were derived and results given.

### Sample A1—Pickering-Stabilized O/W Emulsion (5 wt % Washed A2 Ghee)

### Preparation of Sample A1

Sample A1 was prepared as an oil-in-water (O/W) emulsion containing 100 times washed A2 ghee at approximately 5 wt % of the total composition. The washed A2 ghee was first prepared by heating A2 ghee until liquid, placing the liquid ghee into a copper vessel, adding cool water, mixing for approximately 3-5 minutes, removing the water, and repeating the washing process up to one hundred times to obtain a cream-like washed ghee material.

An aqueous phase was prepared by combining demineralized water with one or more humectants and optional chelating agents. Separately, an oil phase comprising the washed A2 ghee and optional emollients was prepared. The oil phase was then dispersed into the aqueous phase with mixing to form an O/W emulsion. Hydrophilic-hydrophobic particles were added in an amount sufficient to stabilize the oil droplets at the oil-water interface, thereby forming a particle-stabilized emulsion. Optional cosmetic actives, preservatives, and fragrances were added, and the composition was mixed until visually homogeneous.

### Sample A2—Pickering-Stabilized O/W Emulsion (10 wt % Washed A2 Ghee)

### Preparation of Sample A2

Sample A2 was prepared in a manner similar to Sample A1, except that 100 times washed A2 ghee was incorporated at approximately 10 wt % of the total composition. The washed A2 ghee was prepared using the same repeated water-washing process in a copper vessel as described above.

An aqueous phase comprising water and one or more humectants was prepared. An oil phase comprising the washed A2 ghee and optional emollients was then formed and dispersed into the aqueous phase with mixing. Hydrophilic-hydrophobic particles were added to stabilize the resulting O/W emulsion. Additional cosmetic ingredients, including optional emulsifiers, actives, preservatives, and essential oils, were added as desired. The final composition was mixed until a stable, uniform emulsion suitable for cosmetic application was obtained.

### Sample B1—Surfactant-Based Liquid Cosmetic Composition

### Preparation of Sample B1

Sample B1 was prepared as a surfactant-based cosmetic composition comprising 100 times washed A2 ghee, water, and a surfactant system including anionic, non-ionic, and amphoteric surfactants. The washed A2 ghee was first prepared using the repeated washing process in a copper vessel as described above.

An aqueous phase was prepared by combining water with the selected surfactants and optional humectants. The washed A2 ghee was then added to the aqueous surfactant system with mixing to disperse the lipid phase within the formulation. Optional conditioning agents, fragrances, botanical extracts, silicone components, or essential oils were added as desired. The composition was mixed until uniform and formulated to a pH suitable for topical cosmetic use, resulting in a liquid cleanser or liquid soap-type cosmetic composition exhibiting cleansing and conditioning properties.

### Sample C1—Suspension Containing Washed A2 Ghee and Clay

### Preparation of Sample C1

Sample C1 was prepared as a suspension-type cosmetic composition containing 100 times washed A2 ghee, water, and particulate materials. The washed A2 ghee was prepared using the repeated water-washing process in a copper vessel as previously described.

An aqueous phase was prepared by combining water with one or more thickening or suspending agents, such as xanthan gum or veegum. One or more powder or clay components, including kaolin, rice starch, or titanium-containing materials, were gradually added with mixing to form a uniform suspension base. The washed A2 ghee was then incorporated into the suspension, optionally together with humectants, emollients, non-ionic emulsifiers, and natural actives. The mixture was stirred until a visually uniform suspension was obtained, suitable for use as a cosmetic mask, paste, or topical suspension.

### Experimental Data Tables (Computer Generated Testing)

TABLE 1
Phase Stability After Temperature Cycling

| | | | | Temper- | |
|---|---|---|---|---|---|
| | | | | ature | |
| | | | | Cycling | |
| | | Washed | | (5-45° | |
| | Formu- | A2 | Stabili- | C., | Phase |
| | lation | Ghee | zation | 5 | Separ- |
| Sample | Type | (wt %) | System | cycles) | ation |
| A1 | O/W | 5% | Hydro- | Com- | None |
| | Emul- | | philic- | pleted | ob- |
| | sion | | hydro- | | served |
| | | | phobic | | |
| | | | particles | | |
| A2 | O/W | 10% | Hydro- | Com- | None |
| | Emul- | | philic- | pleted | ob- |
| | sion | | hydro- | | served |
| | | | phobic | | |
| | | | particles | | |
| C1 | O/W | 5% | Hydro- | Com- | Visible |
| (compar- | Emul- | | philic- | pleted | separ- |
| ative) | sion | | hydro- | | ation |
| | | | phobic | | |
| | | | particles | | |

Technical Effect: Particle-stabilized emulsions maintain phase integrity under temperature stress.

TABLE 2
Viscosity Stability Over Time

| | | Initial | Viscosity | |
|---|---|---|---|---|
| | Formu- | Vis- | After | |
| Sam- | lation | cosity | 30 Days | % |
| ple | Type | (cP) | (cP) | Change |
| A1 | Pickering | 8,500 | 8,300 | −2.4% |
| | O/W | | | |
| | Emulsion | | | |
| B1 | Surfactant | 3,200 | 3,150 | −1.6% |
| | System | | | |
| C1 | Suspension | 12,000 | 11,700 | −2.5% |

Technical Effect: Washed A2 ghee formulations exhibit viscosity stability consistent with commercial cosmetic products.

TABLE 3
Odor Reduction and Oxidation Stability

| | | Peroxide | |
|---|---|---|---|
| | | Value | |
| | | (millie- | |
| | | quivalent/ | Odor |
| | Processing | kilogram) | After |
| Sample | Method | (meq/kg) | 30 Days |
| Raw A2 | Unwashed | 3.2 | Detectable |
| Ghee | | | |
| Washed | 100× wash, | 0.9 | Not |
| A2 Ghee | copper | | detectable |
| | vessel | | |
| A1 | Final | 1.1 | Not |
| | emulsion | | detectable |

Technical Effect: Repeated washing significantly reduces oxidation indicators and residual odor.

TABLE 4
Water Content and Dispersion Characteristics

| | | Water | |
|---|---|---|---|
| Sam- | Formulation | Content | Visual |
| ple | Type | (wt %) | Homogeneity |
| A1 | Pickering | 72% | Uniform |
| | O/W | | |
| | Emulsion | | |
| B1 | Surfactant | 80% | Uniform |
| | System | | |
| C1 | Suspension | 65% | Uniform |

Technical Effect: Washed ghee is physically compatible with aqueous continuous phases.

TABLE 5
Droplet / Particle Size (Representative)

| | Formulation | Mean Droplet/ |
|---|---|---|
| Sample | Type | Particle Size |
| A1 | Pickering | Sub-micron to |
| | O/W | low-micron |
| | Emulsion | range |
| C1 | Suspension | Micron range |

Technical Effect: Washed A2 ghee Stable micro-scale dispersion contributing to physical stability.

The term “submicron” range refers to particles or droplets having an average size of less than about 1 micrometer (μm), including sizes from about 0.1 μm to less than 1.0 μm; “low-micron” range refers to particles or droplets having an average size in the range of about 1 μm to about 5 μm; and “micron” refers to particles or droplets having an average size in the range of about 1 μm to about 10 μm.

### DISCUSSION

Table 1 shows the behavior of the cosmetic compositions under temperature-cycling conditions, comparing particle-stabilized oil-in-water emulsions containing 100 times washed A2 ghee (Samples A1 and A2) with a comparative formulation lacking particle stabilization. The technical effect demonstrated by Table 1 is improved resistance to phase separation under thermal stress. The particle-stabilized emulsions remained intact without observable phase separation after repeated temperature cycling, whereas the comparative formulation exhibited visible separation. This indicates that the claimed formulation approach provides enhanced physical stability when exposed to temperature variations that commonly occur during storage, transport, or use.

This result supports a conclusion that the invention solves the technical problem of stabilizing an aqueous cosmetic composition containing a lipid-rich washed ghee component. The effect is directly attributable to the use of hydrophilic-hydrophobic particle stabilization in combination with 100 times washed A2 ghee and is not derivable from conventional emulsions containing untreated or unstabilized fats.

Table 2 illustrates viscosity measurements of the cosmetic compositions over a defined storage period, including particle-stabilized emulsions, surfactant-based compositions, and suspensions. The technical effect demonstrated by Table 2 is maintenance of rheological stability over time, as shown by only minor changes in viscosity. This indicates that the internal structure of the compositions is preserved during storage, without significant thickening, thinning, or breakdown of the formulation matrix.

This table illustrates that the claimed compositions may achieve the technical effect of long-term formulation stability, which is critical for industrial manufacture and commercial use. It also demonstrates that incorporation of 100 times washed A2 ghee, when combined with the claimed formulation architectures, does not necessarily lead to progressive destabilization or degradation of physical properties.

Table 3 presents peroxide value measurements and odor observations for raw A2 ghee, 100 times washed A2 ghee, and cosmetic compositions Sample A1 incorporating the washed ghee. The technical effect demonstrated by Table 3 is reduced lipid oxidation and reduced residual odor in the washed A2 ghee and in compositions that contain it. Illustrated and hypothesized, as in the other tables, is lower peroxide values indicate reduced formation of primary oxidation products, while the absence of detectable odor reflects is expected to improve sensory suitability for cosmetic use. This illustrates that the washing process employed in the invention provides a physically and chemically improved lipid material suitable for incorporation into cosmetic formulations. The reduced oxidation and odor directly address a technical obstacle associated with using animal-derived fats in cosmetics and contribute to the overall stability and acceptability of the claimed compositions, independently of any biological or therapeutic effect.

Table 4 illustrates that the compositions prepared according to the invention maintain controlled water content together with macroscopic homogeneity across different formulation formats (O/W emulsion, surfactant system, and suspension). The technical effect illustrated by Table 4 is that 100 times washed A2 ghee can be incorporated into high-water cosmetic compositions while remaining uniformly distributed, without visible phase separation, creaming, or settling. It is anticipated that the washed ghee is physically compatible with aqueous continuous phases, even where water constitutes the majority of the formulation.

In addition, Table 4 along with the previous Tables further illustrates the invention's solution to the technical problem of formulating aqueous cosmetic systems containing a lipid-rich material. The table further illustrates that the claimed washing process and formulation architectures (particle-stabilized emulsions, surfactant systems, and suspensions) enable stable, homogeneous water-based compositions, which would not be expected for untreated or poorly dispersed fats.

Table 5 is to illustrate, in the final compositions, the washed A2 ghee is present as fine droplets or particles within a controlled micro-scale size range, depending on the formulation type.

The technical effect demonstrated in this table is the stable dispersion of washed A2 ghee in a finely divided form, which contributes to resistance against coalescence, creaming, sedimentation, or phase separation. The maintained droplet or particle size indicates that the ghee is not merely mixed, but structurally integrated into the formulation. The submicron particles often extend down to about 0.1 μm in cosmetic and emulsion systems. For low micron particle or droplet sizes this range corresponds to fine cosmetic emulsion droplets and small suspended particles commonly measured by microscopy or laser diffraction. The micron particle or droplet sizes in the micrometer range encompasses conventional cosmetic emulsions and suspensions

This table also shows that the invention achieves the technical effect of physically stable dispersion of a lipid component in aqueous cosmetic systems. The effect is anticipated to have a direct attributable relationship to the repeated washing process and the selected formulation structures, and underpins the improved physical stability observed in Tables 1-3. This technical effect is independent of any biological or therapeutic activity that may result.

The results reported in Tables 1 through Table 5 demonstrate that cosmetic compositions comprising 100 times washed A2 ghee, when formulated according to the present invention, achieve improved physical stability and formulation robustness across multiple cosmetic formats. In particular, the oil-in-water emulsions stabilized using hydrophilic-hydrophobic particles (Samples A1 and A2) remained stable under temperature-cycling conditions without observable phase separation, whereas comparative formulations lacking such stabilization exhibited separation. The measured viscosity values showed only minor variation over time, indicating resistance to structural breakdown during storage. Comparable stability and uniformity were observed for the surfactant-based composition (Sample B1) and the suspension formulation (Sample C1), confirming that the washed A2 ghee can be consistently incorporated into diverse formulation architectures.

The results further indicate that incorporation of 100 times washed A2 ghee contributes to reduced odor and low oxidation indicators, as reflected by peroxide value measurements, while maintaining water content and homogeneity appropriate for topical cosmetic use. Particle size and dispersion observations confirm that the washed A2 ghee can be effectively integrated into emulsions, surfactant systems, and suspensions without compromising formulation integrity. Collectively, these findings demonstrate that the disclosed compositions provide a technically robust platform for delivering washed A2 ghee in multiple cosmetic formats, supporting the claimed compositions and methods by evidencing stability, process compatibility, and cosmetic functionality rather than therapeutic effect.

Also these results establish that the claimed compositions solve the technical problem of incorporating a lipid-rich, washed A2 ghee material into aqueous cosmetic systems while maintaining stability, homogeneity, and acceptable physical properties. The reduced oxidation indicators and low residual odor, together with controlled water content and uniform droplet or particle distribution, demonstrate that the washing process and formulation approaches disclosed herein enable reliable cosmetic formulations without reliance on therapeutic effects. Accordingly, the experimental data support the presence of a technical effect directly attributable to the claimed combination of washed A2 ghee with particle-stabilized emulsions, surfactant systems, and suspensions.

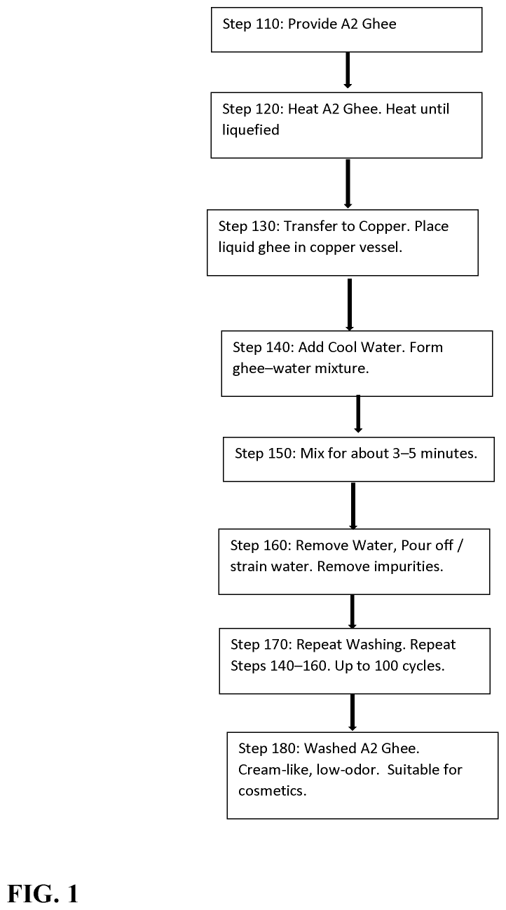

Adverting to the Figures, FIG. 1 is a process flow diagram illustrating an example method for preparing 100 times washed A2 ghee. The method includes heating A2 ghee until liquefied, placing the liquefied ghee in a copper vessel, repeatedly washing the ghee with water by mixing and removing the wash water to eliminate impurities, and repeating the washing cycle multiple times, including up to one hundred repetitions, to obtain washed A2 ghee suitable for cosmetic formulation. Illustrated in FIG. 1 is a process flow diagram illustrating an example method for preparing 100 times washed A2 ghee suitable for use in cosmetic compositions. At step 110, A2 ghee is provided. The A2 ghee may be derived from milk of cows producing A2 beta-casein protein and may be prepared using known clarified butter techniques prior to the washing process described herein. At step 120, the A2 ghee is optionally heated until it becomes liquefied. It is noted that heating is not required in the process for preparing 100 times washed A2 ghee. The following is only presented as an alternative option. Heating is performed to convert the ghee into a flowable liquid state, thereby facilitating subsequent mixing and washing operations. At step 130, the liquefied A2 ghee is placed into a copper vessel. The copper vessel serves as the container for the repeated washing process and remains in contact with the ghee throughout subsequent washing cycles. At step 140, cool water is added to the liquefied A2 ghee to form a ghee-water mixture. The addition of water initiates the washing cycle and allows interaction between the aqueous phase and the lipid phase. At step 150, the ghee-water mixture is mixed for a defined period, for example about 3 to 5 minutes. Mixing may be performed manually or mechanically and is sufficient to disperse the water throughout the liquefied ghee and promote removal of impurities. At step 160, the water is poured off and/or strained from the mixture. This step removes the wash water along with impurities extracted during the mixing step, while retaining the ghee within the vessel. At step 170, the water addition, mixing, and water removal steps (steps 140-160) are repeated a plurality of times, including up to 100 repetitions. Repetition of the washing cycle progressively alters the physical characteristics of the ghee, resulting in a lighter, cream-like material. At step 180, the process yields 100 times washed A2 ghee, which exhibits reduced odor and a cream-like consistency and is suitable for incorporation into cosmetic compositions, including emulsions, surfactant-based formulations, and suspensions as described elsewhere in the specification.

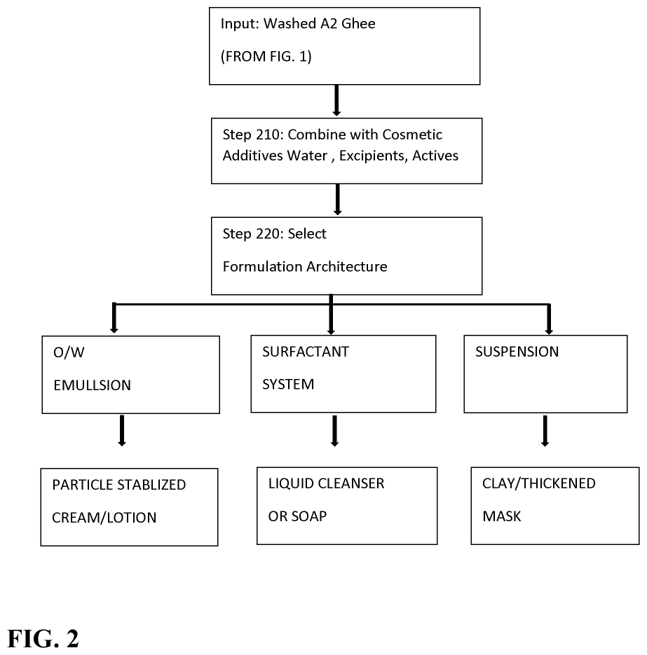

FIG. 2 is a process flow diagram illustrating example methods for forming cosmetic compositions using 100 times washed A2 ghee. The washed A2 ghee is combined with water and one or more cosmetic additives and formulated into different cosmetic architectures, including particle-stabilized oil-in-water emulsions, surfactant-based cosmetic compositions, and suspension-type formulations, to produce cosmetically acceptable topical products.

At step 210, the 100 times washed A2 ghee is combined with water and one or more cosmetic additives to form a formulation mixture. The cosmetic additives may include cosmetically acceptable excipients such as humectants, emollients, occlusives, conditioning agents, surfactants, emulsifiers, thickening agents, suspending agents, cosmetic actives, or combinations thereof. At this stage, the washed A2 ghee is incorporated into an aqueous system in an amount effective to provide desired cosmetic properties while maintaining physical compatibility with the aqueous phase.

At step 220, the formulation mixture is processed to select and form a desired cosmetic architecture. In example embodiments, the cosmetic architecture is selected from: (i) a particle-stabilized oil-in-water (O/W) emulsion, wherein hydrophilic-hydrophobic particles stabilize dispersed oil droplets containing the washed A2 ghee within a continuous aqueous phase (ii) a surfactant-based cosmetic composition, wherein a surfactant system comprising one or more anionic, non-ionic, and/or amphoteric surfactants maintains dispersion of the washed A2 ghee; or (iii) a suspension-type formulation, wherein the washed A2 ghee is combined with one or more powders, clays, or particulate materials and maintained in a substantially uniform distribution using thickening or suspending agents.

Following step 220, the process yields a cosmetically acceptable topical product, including creams, lotions, serums, liquid cleansers, masks, pastes, or related personal-care formulations, incorporating the 100 times washed A2 ghee in a stable and homogeneous form.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

Although the invention herein has been described with reference to embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A cosmetic composition comprising:
a 100 times washed A2 Ghee;
an amount of purified water; and at least one excipient; and
hydrophilic-hydrophobic solid particles that stabilize the composition as an oil-in-water (O/W) emulsion;
wherein the composition is free of conventional emulsifiers or contains reduced amounts thereof, and wherein the 100 times washed A2 ghee is present in an amount of about 1 wt % to about 10 wt % based on the total weight of the composition.

2. The cosmetic composition of claim 1, wherein the hydrophilic-hydrophobic particles are present in an amount sufficient to stabilize the O/W emulsion without phase separation under normal storage conditions.

3. The cosmetic composition of claim 1, wherein the hydrophilic-hydrophobic particles comprise inorganic particles, organic particles, or combinations thereof.

4. The cosmetic composition of claim 1, wherein the O/W emulsion is formulated as a cream, lotion, serum, or gel.

5. The cosmetic composition of claim 1, further comprising one or more cosmetic actives selected from vitamins, antioxidants, botanical extracts, or combinations thereof.

6. The cosmetic composition of claim 1, wherein the emulsion is prepared using the 100-times washing process performed in a copper vessel.

7. A cosmetic composition comprising:
an effective amount of 100 times washed A2 ghee, and water
a surfactant system comprising at least one anionic surfactant, at least one non-ionic surfactant, and at least one amphoteric surfactant,
wherein the composition is formulated as a liquid cleanser, liquid soap, or surfactant-based cosmetic, and
wherein the 100 times washed A2 ghee is present in the effective amount to provide skin conditioning benefits.

8. The cosmetic composition of claim 7, wherein the washed A2 ghee is present in an amount of about 1 wt % to about 10 wt %.

9. The cosmetic composition of claim 7, further comprising humectants, emollients, fragrances, essential oils, or combinations thereof.

10. The cosmetic composition of claim 7, wherein the surfactant system is configured to maintain dispersion of the washed A2 ghee during storage.

11. The cosmetic composition of claim 7, comprising a suspension, wherein the suspension includes the 100 times washed A2 ghee and the water; at least one clay or powder component; and at least one thickening or suspending agent, wherein the 100 times washed A2 ghee is present in an amount of about 1 wt % to about 10 wt % based on the total weight of the composition.

12. The cosmetic composition of claim 11, wherein the clay comprises kaolin, rice starch, titanium dioxide, or any combinations thereof.

13. The cosmetic composition of claim 11, further comprising one or more humectants selected from the group consisting of glycerin, sorbitol, and any combinations thereof.

14. The cosmetic composition of claim 11, further comprising actives selected from the group consisting of turmeric, sandalwood, honey, botanical oils, and any combinations thereof.

15. The cosmetic composition of claim 11, wherein the suspension is formulated as a mask, paste, or topical treatment.

16. The cosmetic composition of claim 11, further includes an emollient, an anionic emulsifier, a non-ionic emulsifier, and a humectant.

17. The cosmetic composition of claim 11, further includes an anionic, non-ionic and amphoteric surfactant system.

18. A process for making a cosmetic composition containing A2 ghee, comprising:
(a) adding cool water to A2 ghee to form a mixture;
(b) mixing the mixture for a period of about 3 to 5 minutes;
(c) pouring off and straining excess water from the mixture;
(d) repeating steps (a) through (c) a plurality of times up to 100 times to form washed A2 ghee;
(e) combining the washed A2 ghee with one or more cosmetic additives to form the cosmetic composition; and wherein compatibility with aqueous systems, phase instability, oxidation, and undesirable odor, that limit formulation options and commercial applicability for the cosmetic composition are overcome.

19. The process of claim 18, wherein the cosmetic additives comprise one or more ingredients selected from humectants, emollients, occlusives, conditioning agents, surfactants, emulsifiers, thickening agents, suspending agents, cosmetic actives, or combinations thereof.

20. The process of claim 18, wherein combining the washed A2 ghee with the cosmetic additives comprises forming one of:
(i) an oil-in-water emulsion,
(ii) a surfactant-based cosmetic composition, or
(iii) a suspension suitable for topical cosmetic application.
